(19) United States
(12) Patent Application Publication
(10) Pub. No.: US 2007/0037179 A1
(43) Pub. Date: Feb. 15, 2007

(54) Methods of diagnosing and prognosticating solid tumors and melanoma

(76) Inventors: Alberto Liboni, Ferrara (IT); Stefania Gessi, Ferrara (IT); Pier Andrea Borea, Ferrara (IT); Steve MacLennan, Cary, NC (US); Edward Leung, Cary, NC (US)

Correspondence Address: JONES DAY, 222 EAST 41ST ST, NEW YORK, NY, 10017, US

(21) Appl. No.: 11/334,332

(22) Filed: Jan. 17, 2006

Related U.S. Application Data
(63) Continuation of application No. 11/129,553, filed on May 13, 2005, now abandoned.
(60) Provisional application No. 60/571,672, filed on May 14, 2004.

(51) Int. Cl. C12Q 1/68; G01N 33/574

(52) U.S. Cl. 435/6; 435/7.23

(57) ABSTRACT

The invention relates to the diagnosis and prognosis of solid tumors and melanoma by detecting increased cellular levels of A₃ receptors in tissue and/or leukocytes obtained from patients with cancer or patients at risk for developing cancer.

### CROSS-REFERENCE TO RELATED APPLICATIONS

This application claims the benefit of priority of U.S. provisional application Ser. No. 60/571,672, filed May 14, 2004; the disclosure of which is hereby incorporated by reference in its entirety.

### FIELD OF INVENTION

The invention relates to the diagnosis and prognosis of solid tumors and melanoma by detecting increased cellular levels of A₃ receptors in tissue and/or leukocytes obtained from patients with cancer or patients at risk for developing cancer.

### BACKGROUND OF THE INVENTION

Adenosine, a ubiquitous nucleoside released from metabolically active or stressed cells is known to act as an important regulatory molecule through its activation of cell surface receptors named A₁, A_{2A}, A_{2B} and A₃, all of which belong to the G-protein coupled superfamily of receptors (1). In particular A₁ and A₃ inhibit, through Gi proteins, adenylyl cyclase activity, whereas and A_{2A} and A_{2B} stimulate, via Gs proteins, this enzyme (2). Collectively, these receptors are widespread on virtually every organ and tissue and represent promising drug targets for the pharmacological intervention in many pathophysiological conditions that are believed to be associated with changes of adenosine levels such as asthma, neurodegenerative disorders, chronic inflammatory diseases and cancer (3).

In solid tumors, chronic hypoxia is observed, due to an insufficient vascularization and the limited diffusion of oxygen into the tissue (4). In this context adenosine, derived from a decrease of cellular ATP, is released into the extracellular space and may have a significant influence on the vasculature, on the resistance to immune attack and on the growth of tumor masses. The immunosuppressive and anti-inflammatory effects of adenosine together with its angiogenic actions strongly suggest that adenosine receptors could be involved in tumorigenesis (5-7). In addition a number of studies have now reported a pivotal role of adenosine on the regulation of cell cycle, proliferation and apoptosis in cells of both tumor (8-12) and nontumor origin (13). These effects depend on the extracellular concentration of adenosine, expression of different adenosine receptor subtypes and the signal transduction mechanisms activated following the binding of specific agonists.

Several lines of evidence indicate the A₃ receptor as the principle subtype responsible for the adenosine-induced inhibition of tumor cell proliferation (11,12,14). Indeed, a dual effect in colon carcinoma-bearing mice, that is, the induction of anticancer activity concomitantly with a myeloprotective effect, has been demonstrated for A₃ receptor activation. The anticancer activity was attributed to direct antiproliferative effect and the myeloprotection to an indirect effect manifested by upregulation of IL12 and natural killer cell activity (15). In support of the A₃ receptor involvement in tumors, it has recently been shown that A₃ receptors are highly expressed on the cell surface of tumor cells (16-19), but not in the majority of normal tissues (14). However, despite promising in vitro and animal studies concerning the A₃-mediated reduction of tumor growth (20), convincing evidence of the presence of this adenosine subtype on solid mirrors is largely missing. These observations constitute a rationale for studying the expression of A₃ receptors in a very common and lethal malignancy such as colorectal cancer.

Colon cancer is the second most frequently diagnosed malignancy in the United States as well as the second most common cause of cancer death. An estimated 95,600 new cases of colon cancer will be diagnosed in 1998, with an estimated 47,700 deaths. The five-year survival rate for patients with colorectal cancer detected in an early localized stage is 92%; unfortunately, only 37% of colorectal cancer is diagnosed at this stage. The survival rate drops to 64% if the cancer is allowed to spread to adjacent organs or lymph nodes, and to 7% in patients with distant metastases.

The prognosis of colon cancer is directly related to the degree of penetration of the tumor through the bowel wall and the presence or absence of nodal involvement, consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy. Recurrence following surgery (the most common form of therapy) is a major problem and is often the ultimate cause of death. In spite of considerable research into therapies for the disease, colon cancer remains difficult to diagnose and treat.

Despite major advances in uncovering the basic biochemical and genetic alterations involved in the development and progression of colorectal cancers, treatment of this disease still relies predominantly upon surgical resection. Moreover, patients' prognosis is determined primarily by the stage of disease at the time of diagnosis (21). Thus, a better understanding of the molecular proteins involved in colorectal cancer cell proliferation would greatly facilitate both the discovery of new possible diagnostic and prognostic markets and the development of novel therapeutic agents.

### SUMMARY OF THE INVENTION

The invention relates to the diagnosis and prognosis of solid tumors and melanoma by detecting increased cellular levels of A₃ receptors in tissue and/or leukocytes obtained from patients with cancer or patients at risk for developing cancer. In some embodiments the invention relates to the diagnosis and prognosis in mammals (including humans) of solid tumors including, but not limited to, pancreatic carcinoma, breast carcinoma, prostate carcinoma, colorectal carcinoma, lung carcinoma and ovarian carcinoma. In other embodiments the invention relates to the diagnosis and prognosis in mammals (including humans) of melanoma. By way of embodiment, the methods and kits described herein relate to the diagnosis and prognosis in mammals of colorectal carcinoma, as this cancer type serves as a prototypical model of malignant lethality. Those of skill in the art will recognize applications of the methods and kits described herein for diagnosis and prognosis of other cancer types in mammals.

In one embodiment, the invention relates to a method for the diagnosis of cancer in a mammal, comprising
(a) obtaining a biological sample from the mammal; and
(b) detecting overexpression of A₃ receptor comprising the polypeptide of SEQ ID NO: 1 in the sample compared to the level in a healthy mammal, wherein said increased level is diagnostic of having cancer in the mammal. The biological sample can include, but is not limited to leukocytes, typically derived from blood or tissue obtained by biopsy (e.g., tissue from a body site suspected of containing a tumor or skin in patients suspected of suffering from melanoma).

In some embodiments, the cancer is a solid tumor. Non-limiting examples of such tumors are pancreatic carcinoma, breast carcinoma, prostate carcinoma, colorectal carcinoma, lung carcinoma and ovarian carcinoma. In other embodiments the cancer is melanoma.

The methods for determining the overexpression of A₃ receptors include a detectably labeled ligand-receptor binding assays, detectably labeled immunocytochemical assays, detectably labeled flow cytometric techniques and RT-PCR.

In another embodiment, the invention relates to a method for the diagnosis of cancer in a mammal, comprising
(a) obtaining a biological sample from the mammal; and
(b) detecting an overexpression of A₃ receptor protein in the sample compared to the level in a healthy mammal by using an detectably labeled antibody that specifically binds the polypeptide of SEQ ID NO: 1 wherein said increased level is diagnostic of cancer in the mammal.

In still another embodiment, the invention relates to a method for the diagnosis of colorectal cancer in a mammal, comprising
(a) obtaining a biological sample from the mammal; and
(b) detecting an overexpression of A₃ receptor protein in the sample compared to the level in a healthy mammal by using a detectably labeled ligand that specifically binds the polypeptide of SEQ ID NO: 1 wherein said increased level is diagnostic of cancer in the mammal.

Other and further aspects, features and advantages of the present invention will be apparent from the following description of the presently preferred embodiments of the invention given for the purpose of disclosure.

### BRIEF DESCRIPTION OF THE FIGURES

FIG. 1 represents a depiction of saturation curves of [³H]MRE 3008-F20 binding to A₃ adenosine receptors in paired carcinomatous (▪) and normal (▾) colon membranes (N=73) (upper panel); scatchard plots of specific binding for calculation of ligand affinity (K_{D}) and maximal number of binding sites (B_{MAX}) (lower panel); data are the mean of different individual experiments; (*P<0.05, ANOVA followed by Dunnett's test).

FIG. 2 represents a depiction of histograms which show the increase of K_{D} (upper panel) and B_{MAX} values (lower panel) of [³H]-MRE 3008F20 binding to human A₃ adenosine receptors in lymphocytes and neutrophils obtained from patients with colorectal cancer (LTC and NTC, respectively) (N=30) with respect to control subjects (L and N, respectively) (N=20). (*P<0.01, Student's t-test); the longitudinal analysis of A₃ receptor density and affinity in a cohort of patients (N=10) studied after 12 months from surgical resection is also included (SRLTC and SRNTC in lymphocytes and neutrophils, respectively); (**P<0.01 versus corresponding TC, Student's t-test).

FIG. 3 is a photographic representation of A₃ adenosine receptor immunostaining: strong reactivity of a moderately differentiated adenocarcinoma (upper panel, A); corresponding normal mucosa distant from the tumor showed weak staining prevalently localized in superficial epithelial cells (lower panel, B); (Immunoperoxidase. Original magnification: A) 400×; B) 100×).

FIG. 4 represents a depiction of histograms showing the same level of A₃ adenosine receptor mRNA levels in all paired carcinomatous and normal colon tissues examined; the mRNA content of each sample was first expressed as A₃ receptor mRNA/β-actin mRNA, then calculated as the ratio of carcinomatous/normal.

FIG. 5 represents the human amino acid sequence of the A₃ receptor (SEQ ID NO:1; Swissprot accession number P33765).

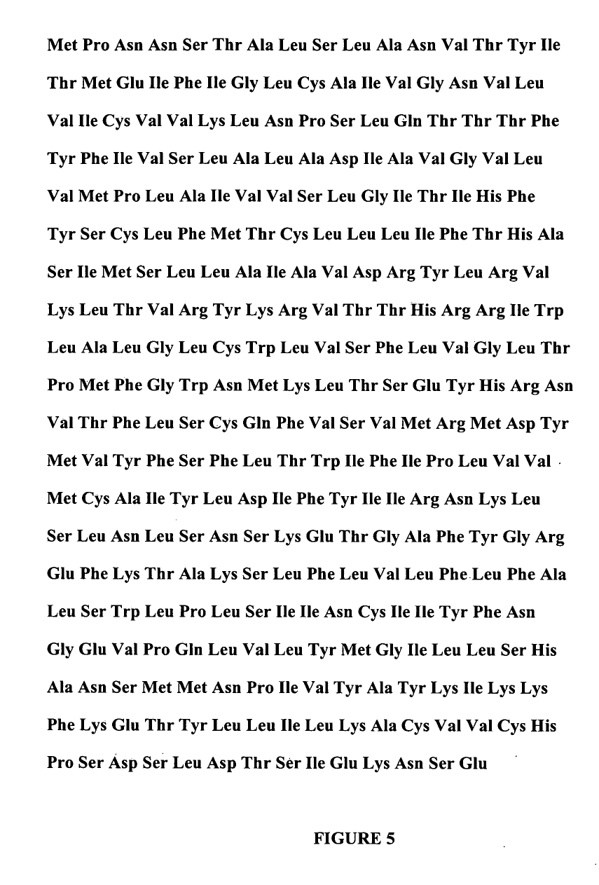

### DETAILED DESCRIPTION OF THE INVENTION

The invention relates to the diagnosis and prognosis of solid tumors and melanoma by detecting increased cellular levels of A₃ receptors in tissue and/or leukocytes obtained from patients with cancer or patients at risk for developing cancer. In some embodiments the invention relates to the diagnosis and prognosis in mammals (including humans) of solid tumors including, but not limited to, pancreatic carcinoma, breast carcinoma, prostate carcinoma, colorectal carcinoma, lung carcinoma and ovarian carcinoma. In other embodiments the invention relates to the diagnosis and prognosis in mammals (including humans) of melanoma. By way of embodiment, the methods and kits described herein relate to the diagnosis and prognosis in mammals of colorectal carcinoma, as this cancer type serves as a prototypical model of malignant lethality. Those of skill in the art will recognize applications of the methods and kits described herein for diagnosis and prognosis of other cancer types in mammals.

In a specific embodiment, the present invention relates to methods for the diagnosis and prognosis of colorectal carcinoma by detecting increased cellular levels of A₃ receptors in colorectal tissue, neutrophils and lymphocytes obtained from patients with colorectal cancer or patients at risk for developing colorectal cancer.

Herein, “biological sample” means a sample of cells from a patient. These cells may be part of a cancerous tissue or organ sample obtained, for example, by biopsy, or they may be individual cells, for example, blood cells or even cells grown in culture. Preferably, such cells are cells obtained from peripheral blood. The cells from the blood that are assayed can be any or all leukocyte cells present in the blood. Such cells are commonly obtained by drawing a blood sample from a patient and then using standard techniques to purify or partially purify the cells from the blood. For example, a cellular fraction can be prepared as a “buffy coat” (i.e., leukocyte-enriched blood portion) by centrifuging whole blood for a short time at low speed (e.g., 10 min at 800 times gravity) at room temperature. Red blood cells sediment most rapidly and are present as the bottom-most fraction in the centrifuge tube. The buffy coat, containing the leukocytes, is present as a thin creamy white colored layer on top of the red blood cells. The plasma portion of the blood forms a layer above the buffy coat. Fractions from blood can also be isolated in a variety of other ways. One method is by taking a fraction or fractions from a gradient used in centrifugation to enrich for a specific size or density of cells.

The biological samples may be of normal cells, may be of melanoma cells, or may be of tumor cells, wherein the melanoma or tumor cells are benign or malignant. Generally, an assay that uses cells from such biological samples, herein, will be used to determine the presence of A₃ receptor transcripts or proteins, or levels of A₃ receptor transcripts or proteins. When such an assay is performed, the “test sample” will generally be a sample for which the presence or level of A₃ receptor is unknown and is being tested to provide, for example, an indication of the aggressiveness of the tumor cells. In such an assay, a “control sample” will preferably also be used. The control sample can be from normal (i.e., non-tumorigenic or non-neoplastic) tissue from the same patient from which the test sample is taken or can be from another person known or thought not to have the tumor that is present or thought to be present in the patient from whom the test sample is taken. Preferably, the control sample comprises the same type of cells that comprise the test sample. For example, if the test sample comprises leukocytes, it is preferable that the control sample is also a leukocyte sample.

The biological samples can be obtained from patients at various times. For example, a sample may be obtained from an individual who is suspected of having a tumor or cancer. Assay of such sample using the methods described below can indicate whether the individual has a tumor or cancer. Samples may also be obtained from an individual known to have a tumor or cancer (i.e., the sample is taken after the patient has already been diagnosed). Assay of such sample using the described methods may have prognostic value to the individual. Multiple samples can also be taken from the same individual, for example, at different times after diagnosis. Assay of such samples can indicate whether the cancer or tumor is growing or spreading. Such assays on multiple samples are especially informative in the case where it is desired to determine the effect of a chemotherapeutic, other therapeutic agents or surgical resection on the growth and progression of the tumor or cancer in the individual.

The test samples are preferably obtained from patients who are known to have or suspected of having a tumor or cancer. Methods for diagnosis of particular tumors or cancers in patients are well known in the art of medicine, oncology and hematology.

Herein, “assaying,” when used in reference to biological samples, preferably the cells in biological samples, refers to assessment or measurement of the presence and/or levels or concentrations of A₃ receptor gene expression (transcripts or protein isoforms) in the samples. This assessment is done by detecting and/or measuring the levels of RNA transcribed from the A₃ receptor gene or proteins which are translated from the RNA transcripts. As will be described subsequently, multiple transcripts, called alternative transcripts in the art, may be present when the A₃ receptor gene is transcribed. Such alternative transcripts come from different combinations of exons encoded by the A₃ receptor gene. Some or all of these transcripts are translated to produced A₃ receptor proteins. The A₃ receptor proteins that are obtained from translation of different alternative transcripts, may be somewhat different (in size and/or sequence) from one another depending on the combination of exons existing in the particular alternative transcript or on how the exon sequences in the transcripts are translated. Such different proteins are known in the art as protein “isoforms.”

Assaying these samples, with respect to A₃ receptor expression, may involve detection or quantification of one or more specific alternative transcripts or protein isoforms. For example, one may be interested in determining the presence, level or concentration of one specific alternative transcript or protein isoform. Alternatively, assaying the samples may involve detection or levels of A₃ receptor transcripts or proteins as a whole. For example, in determining the level or concentration of A₃ receptor transcripts, the sum of the levels of all of the different alternative transcripts or protein isoforms may be used.

With regard to elevated levels or elevated concentrations of one or more A₃ receptor alternative transcripts or protein isoforms, “elevated” means an increase in the amount of the transcript or isoform in the test sample as compared to the control sample. “Elevated in the test sample as compared to the control sample” describes a situation where the presence of A₃ receptor transcripts or proteins is detected in the test sample and the amount, level or concentration of the A₃ receptor transcripts or proteins in the test sample is greater than in the control sample. This means, in the control sample, that A₃ receptor transcripts or proteins are detected, but are not present in amounts, levels or concentrations as high as are present in the test sample.

Therefore, to ascertain whether the test sample contains “overexpressed” levels of A3 receptor, a comparison of the levels in the test sample to the levels in one or more control samples is performed. Levels in a control sample or samples can be represented by a single value or range of values. Preferably, an average of the A₃ receptor levels in more than one control sample is used for comparison with the A₃ receptor levels in the test sample. More preferably, an average of the A₃ receptor levels from a number of control samples sufficient to provide a statistically significant comparison with A₃ receptor levels present in the test sample is used. The control sample levels of A₃ receptor may be determined at the same time at which A₃ receptor levels in the test sample is determined. The A₃ receptor levels in the control samples may also be predetermined, meaning that the levels have been determined before the time at which A₃ receptor levels in the test samples are determined. In the case where A₃ receptor levels in control samples are predetermined, the values are preferably normalized or standardized such that they can be legitimately compared with values for A₃ receptor levels in test samples that are determined later.

With reference to overexpressed, increased or elevated levels of A₃ receptor transcripts or proteins in the test sample, the amount of the increase can be of various magnitudes. The increase may be relatively large. For example, a large increase could be a 100% or more increase in A₃ receptor expression in the test sample as compared to the control sample. However, the increase may be relatively small. For example, the increase may be less than 100%, less than 50%, or even less than a 10% increase of the transcript or protein in the test sample as compared to the control sample. Preferably, whatever the degree or magnitude of the increase, such increase is statistically significant. Methods for determining whether an increase is statistically significant are well known in the art of statistics and probability.

Comparison of the test sample to the control sample for the presence and/or levels of A₃ receptor expression is used to characterize the tumor or cancer, i.e., to determine the “aggressiveness” of the tumor or cancer. A level of A₃ receptor transcripts or proteins in the test sample that is higher than the level in the control sample indicates presence of an aggressive tumor or cancer. The extent or degree of the increase between the level of A₃ receptor transcripts or proteins in the test sample and the control sample correlates with degree of aggressiveness of the tumor or cancer. Aggressiveness refers to the nature of tumor cell growth in a patient. For example, an aggressive cancer has a higher probability of producing an unfavorable outcome in a patient than a cancer that is less aggressive. “Unfavorable outcome” normally refers to the probability that a patient will have a relatively short lifespan due to the aggressive nature of the cancer. Patients with a less aggressive cancer or cancer that is not aggressive are expected to have a longer lifespan than a patient with an aggressive form of the cancer.

In some embodiments, the methods of the invention further relate to diagnosing and treating a cancer in a patient (described infra). In such embodiments, treatment can include administering an A₃ receptor antagonist to the patient. Herein, an “effective amount” of the A₃ receptor antagonist refers to an amount of the A3 antagonist sufficient to reduce the ability of adenosine to protect the cancerous (e.g., tumor) cells.

A₃ Receptor Overexpression Associated with Cancers.

Methods are provided to determine the level of A₃ receptor gene expression in a biological sample. One method determines the amount of RNA transcribed from the A₃ receptor gene. Other method determines the amount of A₃ receptor protein, including detectably labeled ligand-receptor binding assays, detectably labeled immunocytochemistry and detectably labeled flow cytometric techniques.

Isolation of RNA from Patient Cell Samples:

To determine the amount of A₃ receptor RNA in a sample, RNA is first isolated from the tissue or cells comprising the biological sample. The biological sample is preferably a biopsy sample of a cancerous tissue (e.g., colon tissue) or a peripheral blood sample containing leukocytes as was described earlier. A variety of methods of RNA isolation from cells and/or tissues is well known to those skilled in the art. Any of such methods can be used. One such method uses the Trizol RTM reagent from Gibco BRL. Such methods isolate total cellular RNA. Other methods isolate polyadenylated RNA. Methods that provide either type of RNA can be used.

RT-PCR:

Reverse transcriptase reactions coupled to polymerase chain reactions (RT-PCR) is one method to assay for the presence of an RNA in a pool of total RNA from a tissue or cell. Detection of a particular RNA is dependent on primers used in the PCR reaction.

RT:

The initial step in RT-PCR is a reverse transcription step. Procedures for reverse transcription are well known to those skilled in the art and a variety of procedures can be used. Either total RNA or polyadenylated mRNA can be used as the template for synthesis of cDNA by the reverse transcriptase enzyme.

In one embodiment, oligo(dT) is used as the primer in the reverse transcription reaction. Oligo(dT) hybridizes to the poly(A) tails of mRNAs during first strand cDNA synthesis. Since all mRNAs normally have a poly(A) tail, first strand cDNA is made from all mRNAs present in the reaction (i.e., there is no specificity). In another embodiment, specific primers are used in place of oligo(dT) and specific RNAs are reverse transcribed into DNA. The specific primers preferably are complementary to a region near the 3′ end of the RNA in order that full length or nearly full length cDNA is produced. Primer selection is preferably made using the guidelines described below for selection of PCR primers. A number of different primers can be used with good results. Another embodiment can be given by the use of random primers.

Preferably, the reverse transcriptase enzyme used in the reaction is stable at temperatures above 60 degree C., for example, SuperScript II RT (Gibco BRL). However, MMLV reverse transcriptase can also be used. In one embodiment, the reverse transcriptase reaction mixture contains 10 mM Tris (pH 8.3), 40 mM KCl, 1.5 mM MgCl₂, 1 mM dithiothreitol, 200 uM each of dATP, dCTP, dTTP and dGTP, 200 ng of the primer, 10 U of AMV reverse transcriptase from Boehringer Mannheim Biochemicals, and 20 units of RNASIN from Promega.

The disaccharide, trehalose, can be added to the reverse transcriptase reaction. Trehalose is a disaccharide that has been shown to stabilize several enzymes including RT at temperatures as high as 60 degree C. (Mizuno, et al., Nucleic Acids Res. 27:1345-1349, 1999). Trehalose addition allows the use of high temperatures in the reverse transcription reaction (e.g., as high as 60 degree C.). Therefore, trehalose can be added to the reverse transcriptase reaction such that it is present in a final concentration of between 20 to 30%. Preferably, the reverse transcriptase reaction is then performed at a temperature between 35 to 75 degree C., more preferably at a temperature from between 50 to 75 degree, most preferably at a temperature of 60 degree C.

PCR:

Once the reverse transcriptase reaction is carried out, the cDNA produced is amplified by PCR. In one embodiment, the entire RT-PCR reaction is carried out on a standard thermal cycler according to the methods described in the GeneAmp RNA PCR kit obtained from Perkin-Elmer/Cetus, for example. A 0.5 pg sample of total RNA from the cells is used to produce the first strand cDNA. The amplification cycle protocol is as follows: 95 degree C. for 2 minutes, 95 degree C. for 1 minute, 56 degree C. for 1 minute, and 72 degree C. for 2 minutes, through 35 cycles. The annealing temperature depends on the primers used.

In another embodiment, a standard PCR reaction contains a buffer containing 10 mM Tris-HCl (pH 8.3), 50 mM KCl, and 6.0 mM MgCl₂, 200 uM each of dATP, dCTP, dTTP and dGTP, two primers of concentration 0.5 uM each, 7.5 ng/ul concentration of template cDNA and 2.5 units of Taq DNA Polymerase enzyme. Variations of these conditions can be used and are well known to those skilled in the art.

The PCR reaction is preferably performed under high stringency conditions. Herein, “high stringency PCR conditions” refers to conditions that do not allow base-pairing mismatches to occur during hybridization of primer to template. Such conditions are equivalent to or comparable to denaturation for 1 minute at 95 degree C. in a solution comprising 10 mM Tris-HCl (pH 8.3), 50 mM KCl, and 6.0 mM MgCl₂, followed by annealing in the same solution at about 62 degree C. for 5 seconds.

The products of the PCR reaction can be detected in various ways. One way is by agarose gel electrophoresis which involves separating the DNA in the PCR reaction by size in electrophoresis. The agarose gel is then stained with dyes that bind to DNA and fluoresce when illuminated by light of various wavelengths. Preferably the dye used is ethidium bromide and the illumination uses an ultraviolet light.

Primer Selection:

One primer is located at each end of the region to be amplified. Such primers will normally be between 10 to 30 nucleotides in length and have a preferred length from between 18 to 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. Preferably, the length of sequence amplified is between 75 and 250 nucleotides in length.

One primer is called the “forward primer” and is located at the left end of the region to be amplified. The forward primer is identical in sequence to a region in the top strand of the DNA (when a double-stranded DNA is pictured using the convention where the top strand is shown with polarity in the 5′ to 3′ direction). The sequence of the forward primer is such that it hybridizes to the strand of the DNA which is complementary to the top strand of DNA.

The other primer is called the “reverse primer” and is located at the right end of the region to be amplified. The sequence of the reverse primer is such that it is complementary in sequence to a region in the top strand of the DNA. The reverse primer hybridizes to the top strand of the DNA.

PCR primers should also be chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence are preferably chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/-primer3_www.cgi.

Forward and reverse primers can be selected from a variety of regions of the A₃ receptor gene. Actually, a very large number of primers can be designed using the sequence of the A₃ receptor gene and such probes successfully used. Preferably, for PCR amplification of A₃ receptor, the forward primer is designed using a sequence within exon 6 of A₃ receptor and the reverse primer is designed using a sequence within exon 8 of A₃ receptor. Both the forward and reverse primers can also be designed using sequences within exon 8.

Real-Time PCR:

The PCR procedure can also be done in such a way that the amount of PCR products can be quantified. Such “quantitative PCR” procedures normally involve comparisons of the amount of PCR product produced in different PCR reactions. A number of such quantitative PCR procedures, and variations thereof, are well known to those skilled in the art. One inherent property of such procedures, however, is the ability to determine relative amounts of a sequence of interest within the template that is amplified in the PCR reaction.

One particularly preferred method of quantitative PCR used to quantify copy numbers of sequences within the PCR template is a modification of the standard PCR called “real-time PCR.” Real-time PCR utilizes a thermal cycler (i.e., an instrument that provides the temperature changes necessary for the PCR reaction to occur) that incorporates a fluorimeter (i.e. an instrument that measures fluorescence). In one type of real-time PCR, the reaction mixture also contains a reagent whose incorporation into a PCR product can be quantified and whose quantification is indicative of copy number of that sequence in the template. One such reagent is a fluorescent dye, called SYBR Green I (Molecular Probes, Inc.; Eugene, Oreg.) that preferentially binds double-stranded DNA and whose fluorescence is greatly enhanced by binding of double-stranded DNA. When a PCR reaction is performed in the presence of SYBR Green I, resulting DNA products bind SYBR Green I and fluoresce. The fluorescence is detected and quantified by the fluorimeter. Such technique is particularly useful for quantification of the amount of template in a PCR reaction.

For reverse transcription of A₃ receptor RNA, two different primers are preferably alternatively used. The first primer is called a A₃ forward primer and has the sequence 5′- ATGCCTTTGGCCATTGTTG-3′ (SEQ ID NO:2). The second primer is called A3 Reverse primer and has the sequence 5′- ACAATCCACTTCTACAGCTGCCT-3′ (SEQ ID NO:3). For reverse transcription of a housekeeping gene, glucose phosphate isomerase (GPI) the primer preferably is called GPI Exon3R and has the sequence 5′-TCGGTGTAGTTGATCTTCTC-3′ (SEQ ID NO:4).

A preferred variation of real-time PCR is TaqMan MGB (Applied Biosystems) PCR. The basis for this method is to continuously measure PCR product accumulation using a dual-labeled fluorogenic oligonucleotide probe called a TaqMan MGB probe. The “probe” is added to and used in the PCR reaction in addition to the two primers. This probe is composed of a short (ca. 20-30 bases) oligodeoxynucleotide sequence that hybridizes to one of the strands that are made during the PCR reaction. That is, the oligonucleotide probe sequence is homologous to an internal target sequence present in the PCR amplicon. The probe is labeled or tagged with two different fluorescent dyes. On the 5′ terminus is a “reporter dye” and on the 3′ terminus is a “quenching dye.” One reporter dye that is used is called 6-carboxy fluorescein (FAM). One quenching dye that is used is called 6-carboxy tetramethyl-rhodamine (TAMRA). When the probe is intact, energy transfer occurs between the two fluorochromes and emission from the reporter is quenched by the quencher, resulting in low, background fluorescence. During the extension phase of PCR, the probe is cleaved by the 5′ nuclease activity of Taq polymerase, thereby releasing the reporter from the oligonucleotide-quencher and producing an increase in reporter emission intensity. During the entire amplification process the light emission increases exponentially.

The instrument used to detect the fluorescence is preferably an ABI Prism 7700, which uses fiber optic systems that connect to each well in a 96-well PCR tray format. The laser light source excites each well and a CCD camera measures the fluorescence spectrum and intensity from each well to generate real-time data during PCR amplification. The ABI 7700 Prism software examines the fluorescence intensity of reporter and quencher dyes and calculates the increase in normalized reporter emission intensity over the course of the amplification. The results are then plotted versus time, represented by cycle number, to produce a continuous measure of PCR amplification. To provide precise quantification of initial target in each PCR reaction, the amplification plot is examined at a point during the early log phase of product accumulation. This is accomplished by assigning a fluorescence threshold above background and determining the time point at which each sample's amplification plot reaches the threshold (defined as the threshold cycle number or CT). Differences in threshold cycle number are used to quantify the relative amount of PCR target contained within each tube.

Northern Blotting

In addition to RT-PCR, other procedures can be used to detect RNA that is transcribed from the A₃ receptor gene. One such method is known as Northern blot hybridization. In this method, RNA is isolated from tissues from body sites suspected of being diseased (e.g., colorectal tissues) or leukocytes and separated by size using gel electrophoresis. The RNAs in the gel are then transferred to a membrane. After transfer of the RNA to the membrane, a nucleotide probe is labeled and hybridized to the RNA on the membrane. Hybridization of a DNA probe to RNA on the membrane is detected by autoradiography or chemiluminescence.

A variation of Northern blotting, is called slot blotting or dot blotting. In this technique, the isolated RNA is applied directly to a membrane. The nucleotide probe is then labeled and hybridized to the RNA on the membrane. Hybridization is detected by autoradiography or chemiluminescence.

Probes of many different lengths and sequences can be designed and used in Northern blotting experiments to detect A₃ receptor transcripts.

Nuclease Protection Assays

In another embodiment, RNA that is transcribed from the A₃ receptor gene can be detected by performing nuclease protection assays (including both ribonuclease protection assays and S1 nuclease assays) to detect and quantitate specific mRNAs (e.g., mRNAs of a gene described in Table 30). Such assays are described in, for example, Sambrook et al., 2001, Molecular Cloning: A Laboratory Manual, 3^{rd} Ed. Cold Spring Harbor Laboratory Press (Cold Spring Harbor, New York), which is incorporated by reference in its entirety. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

Western Blotting

Protein or cell-free extracts are made from organ tissues (e.g., colorectal tissues) or leukocyte cells. In one method, cells are lysed in 500 μl ice-cold Lysis Buffer (50 mM Tris pH 7.5; 1% Triton X-100; 100 mM NaCl; 50 mM NaF; 200 uM NA₃ VO₄; 10 μg/ml pepstatin and leupeptin) (all chemicals from Sigma Chemical Co., St. Louis, Mo.) for approximately 30 min at 4 C. The cell lysate suspension is then microcentrifuged at 4 C. (14,000 RPM for 10 min). The supernatant is removed and stored at −80 degree C. Proteins are separated using SDS-polyacrylamide gel electrophoresis (SDS-PAGE) through a 6%-7.5% acrylamide gel at 100V. The samples are transferred to an Immobilon-P membrane (Millipore, Bedford, Mass.). Blots are blocked in phosphate buffered saline (PBS: 138 mM NaCl, 15 mM Na₂HPO₄, 1.5 mM KCl, and 2.5 mM KH₂PO₄), containing 5% non-fat dehydrated milk and 0.1% Tween-20 (Sigma Chemical Co., St. Louis, Mo.) overnight at 4 degree C. Blots are incubated for 90-120 min at room temperature in PBS with primary anti-A3 receptor antibody and then washed three times in PBS with 0.1% Tween-20. Blots are then incubated with secondary antibody conjugated to horseradish peroxidase (1:4000 dilution) (Sigma Chemical Co., St. Louis, Mo.) for 1 hour at room temperature and washed again as described above. Signal is visualized by incubating with Super Signal chemiluminescent substrate (Pierce, Rockford, Ill.) and exposing the membrane to Kodak scientific imaging film (Kodak, Rochester, N.Y.).

Antibodies

Another method for detecting and quantifying overexpression of A₃ receptor uses antibodies immunospecific for one or more isoforms of the A₃ receptor protein to detect the protein in extracts or fixed cells, for example. Hereinafter, unless specifically indicated otherwise, “A₃ receptor protein” refers not to a single protein, but to multiple A₃ receptor proteins could be represented by different isoforms. The present invention utilizes antibodies that are immunospecific for the A₃ receptor protein. As used herein, the term “immunospecific” means the antibodies have greater affinity for the A₃ receptor protein than for other proteins. Preferably, the affinity of the antibodies for A₃ receptor protein is many fold greater than their affinity for any other proteins. Most preferably, the A₃ receptor antibodies do not have affinity for any proteins other than A₃ receptor protein.

The term “antibody” encompasses monoclonal antibodies, polyclonal antibodies, multispecific antibodies (e.g., bispecific antibodies), and antibody fragments, so long as they exhibit the desired biological activity or specificity. “Antibody fragments” comprise a portion of a full length antibody, generally the antigen binding or variable region thereof. Examples of antibody fragments include Fab, Fab′, F(ab′)₂, and Fv fragments.

Antibodies raised against A3 receptor are produced by immunizing a host animal with a A3 receptor protein or an antigenic fragment thereof. Suitable host animals for injection of the protein immunogen include, but are not limited to, rabbits, mice, rats, goats, and guinea pigs. Various adjuvants may be used to increase the immunological response of the immunogen or antigen (i.e., the A₃ receptor protein or peptide) in the host animal. The adjuvant used depends, at least in part, on the host species. For example, guinea pig albumin is commonly used as a carrier for immunizations in guinea pigs. Such animals produce heterogeneous populations of antibody molecules, which are referred to as polyclonal antibodies and which may be derived from the sera of the immunized animals. Such sera may be used directly, or the specific antibodies desired can be purified from the sera, using methods well known to those of skill in the art.

Antibodies are also prepared using an oligopeptide having a sequence which is identical to a portion of the amino acid sequence of a A₃ receptor protein isoform. Preferably the oligopeptide has an amino acid sequence of at least five amino acids, and more preferably, at least 10 amino acids that are identical to a portion of the amino acid sequence of a A₃ receptor protein. Such peptides are conventionally fused with those of another protein such as keyhole limpet hemocyanin and antibody is produced against the chimeric molecule. Such peptides can be determined using software programs, for example the MacVector program, to determine hydrophilicity and hydrophobicity and ascertain regions of the protein that are likely to be present at the surface of the molecule.

The term “monoclonal antibody” as used herein, refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site, also called epitope. Furthermore, in contrast to conventional (polyclonal) antibody preparations, which typically include different antibodies directed against different determinants (epitopes), each monoclonal antibody is directed against a single determinant on the antigen.

The monoclonal antibodies to be used in accordance with the present invention may be made by the hybridoma method, first described by Kohler and Milstein (Nature 256:495-497, 1975), in which case the hybridoma cell lines that are obtained secrete the monoclonal antibodies during growth. As is known in the art, hybridomas that secrete monoclonal antibodies are made by injecting mice with the desired antigen. The antigens frequently are peptide antigens which are chosen using similar procedures as described above for selection of peptide antigens for making polyclonal antibodies. After the antigens have been injected into the mice, spleen cells are taken from the immunized mice and are fused to myeloma cells. Clones of fusion cells are then obtained and are screened for production of anti-A3 receptor antibodies.

Various immunoassays may be used for screening to identify antibodies having the desired specificity. These include protocols which involve competitive binding or immunoradiometric assays and typically involve the measurement of complex formation between the respective A3 receptor protein and the antibody.

In order to grow the hybridoma cell lines and obtain the secreted antibodies, the hybridoma cell lines may be grown in cell culture and culture medium containing the monoclonal antibodies collected. Alternatively, the hybridoma cell lines may be injected into, and grown within, the peritoneal cavity of live animals, preferably mice. As the hybridoma cell lines grow within the peritoneal cavity of the animal, the monoclonal antibodies are secreted. This peritoneal fluid, called “ascites,” is collected using a syringe to obtain the monoclonal antibodies. Such antibodies may be of any immunoglobulin class including IgG, IgM, IgE, Iga, IgD and any class thereof.

Antibody preparations may be isolated or purified. An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody may be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Antibodies immunospecific for A₃ receptor are useful for identifying tissues (e.g., colorectal tissues) or leukocyte cells that express A₃ receptor proteins. The diagnostic/prognostic methods comprise the steps of contacting tissues or cells with such antibody and assaying for the formation of a complex between the antibodies and a A₃ receptor protein in the samples. Preferably the cells are permeabilized. Interactions between antibodies and a protein or peptide in the sample are detected by radiometric, colorimetric, or fluorometric means. Detection of the antigen-antibody complex may be accomplished by addition of a secondary antibody that is coupled to a detectable tag, such as for example, an enzyme, fluorophore, or chromophore. Preferably, the detection method employs an enzyme-linked immunosorbent assay (ELISA), Western immunoblot procedure and/or immunoprecipitation and/or flow cytometric techniques.

Detectably Labeled

As used herein, “detectably labeled” has the ordinary meaning in the art. A molecule (e.g., antibody or polynucleotide probe) can be detectably labeled by virtue of containing an atom (e.g., radionuclide), molecule (e.g., fluorescein), or complex, that due to a physical or chemical property, indicate the presence of the molecule. A molecule is also detectably labeled when it is covalently bound or otherwise associated with a “reporter” molecules (e.g., a biomolecule such as an enzyme) that act on a substrate to produce a detectable atom, molecule complex. Detectable labels suitable for use in the present invention include any composition detectable by spectroscopic, photochemical, biochemical, immunochemical, electrical, optical or chemical means. Labels useful in the present invention include biotin for staining with labeled avidin or streptavidin conjugate, magnetic beads (e.g., Dynabeads TM), fluorescent dyes (e.g., fluorescein, fluorescein-isothiocyanate [FITC], Texas red, rhodamine, green fluorescent protein, enhanced green fluorescent protein, lissamine, phycoerythrin, Cy2, Cy3, Cy3.5, Cy5, Cy5.5, Cy7, FluorX [Amersham], SyBR Green I & II [Molecular Probes], and the like), radiolabels (e.g., .sup.3 H, .sup. 125 I, .sup.35 S, sup.14 C, or .sup.32 P), enzymes (e.g., hydrolases, particularly phosphatases such as alkaline phosphatase, esterases and glycosidases, or oxidoreductases, particularly peroxidases such as horse radish peroxidase, and the like), substrates, cofactors, inhibitors, chemiluminescent groups, chromogenic agents, and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels and chemiluminescent labels may be detected using photographic film or scintillation counters, fluorescent markers may be detected using a photodetector to detect emitted light (e.g., as in fluorescence-activated cell sorting). Enzymatic labels are typically detected by providing the enzyme with a substrate and detecting the reaction product produced by the action of the enzyme on the substrate, and calorimetric labels are detected by simply visualizing the colored label. Means of detecting labels are well known to those of skill in the art. Thus, for example, where the label is a radioactive label, means for detection include a scintillation counter, photographic film as in autoradiography, or storage phosphor imaging. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Also, simple colorimetric labels may be detected by observing the color associated with the label.

Immunocytochemistry

Suitable methods of cell or tissue preparation and for binding antibodies to antigens include those used for conventional immunocytochemistry, and are well known in the art. Immunophenotyping (e.g., ICC) techniques are known in the art and well within the capacity of one of ordinary skill to apply. See, e.g., Staines, 1988, J. Histochem. Cytochem. 36:145; Gillitzer et al., 1990, J. Histochem. Cytochem. 38:307; Wagner and Worman, 1988, Stain Technology 63:129; McGovern and Crocker, 1987, Am. J. Clin. Pathol. 88,480; Graham et al., 1991, J. Clin. Pathol. 44:96; MaWhinney et al., 1990, J. Clin. Pathol. 43:591. Typically, the cells of interest are fixed (e.g., in buffered formalin) or permeabilized (e.g., using by agents such as buffered detergent solution, e.g., Tween 20, Triton X, or NP 40 [<0.5% v/v] in Tris- or phosphate based buffers). Fixation or permeabilization is particularly preferred when the target antigen is not an extracellular protein (e.g., when the target is a cytoplasmic protein) to increase the accessibility of the antigen to the antibody. According to normal protocols, following antibody binding to the sample, any unbound antibody is removed in a wash step, e.g., PBS (phosphate buffered saline) or Tris-based buffer with or without non-ionic detergent.

Ligand-Receptor Binding Assays

For A₃-adenosine receptor saturation binding assays, generally, radioligand binding assays can be performed in tubes containing binding buffers (such as, 50 mM TRISHCL, pH 7.4, 10 mM MgCl.sub.2, 0.25% BSA), protease inhibitors (5 μg/ml leupeptin, 5 μg/ml aprotinin, 100 μg/ml bacitracin, and 100 μg/ml benzamidine), cold competitor (A3 binding agent, μM final concentration), [³H] A3 binding agent (1-50 nM). Binding reactions are initiated by adding ug amount of membrane protein (ranging from e.g 60 to 100 μg) in a volume which can range from e.g. 250 ul to 1 ml. All incubations can be carried out at 4° C. if an antagonist radioligand is used or at room temperature if an agonist radioligand is used for an incubation time which allows the reaching of the equilibrium. Free radioligand is separated from bound ligand by rapid filtration through a glass fiber filter using a cell harvester. The filter disks are then washed several times with cold (4 degree C.) binding buffer lacking BSA prior to counting. Samples from tissue biopsies or leukocytes are compared to normal controls.

Kits

In certain embodiments, the invention also provides kits that are useful in diagnosing or prognosticating solid tumors and melanoma. In some embodiments, the kits of the present invention comprise one or more reagents that specifically bind to compounds associated with A3 receptor gene expression (e.g., mRNA, RNA transcripts, gene products, peptides, polypeptides, proteins or protein isoforms). Examples of reagents that the kit can include, but are not limited to, proteins and fragments thereof, antibodies, peptides, proteoglycans, glycoproteins, lipoproteins, carbohydrates, lipids, nucleic acids (e.g., DNA, such as cDNA or amplified DNA, or RNA, such as mRNA), organic or inorganic chemicals, natural or synthetic polymers, small molecules (e.g., metabolites), or discriminating molecules or discriminating fragments of any of the foregoing. Here, a discriminating molecule or fragment is a molecule or fragment that, when detected, indicates presence or abundance of a molecule of interest (e.g., a cDNA, amplified nucleic acid molecule, or protein). The kit may also comprise at least one internal standard to be used in verifying the levels of A₃ receptor expression in the methods of the present invention.

In one embodiment, the invention provides kits comprising nucleic acid probes and/or primers that may or may not be immobilized at an addressable position on a substrate. For instance, the kits may contain nucleic acid probes that hybridize to the A₃ receptor comprising the polypeptide comprising SEQ ID NO: 1. The nucleic acid probes may be detectably labeled.

In one embodiment of the invention, the kits contain reagents that can be used to detect A3 receptor comprising the polypeptide comprising SEQ ID NO: 1 contained in the biological samples obtained from the patient. In accordance with this embodiment, the kit may include antibodies, fragments or derivatives thereof (e.g., Fab, F(ab′)2, Fv, or scFv fragments) that are specific for the A3 receptor. In one embodiment, the antibodies may be detectably labeled.

The kits of the present invention may also include reagents such as buffers, or other reagents that can aid in detecting molecules associated with A₃ receptor expression. Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

Advising the Medical Professional

In another aspect, the methods comprise determining that a patient has a cancer that is likely to respond to treatment with an A₃ receptor antagonist, then advising a medical professional of the treatment option of administering to the patient an effective amount of an A₃ receptor antagonist. In certain embodiments, the cancer can be of the solid tumor type or melanoma type. In certain embodiments, the treatment option may include administration of the A₃ receptor antagonist with other anti-tumor agents, such as anti-angiogenic agents (including adenosine A₂ₐ antagonists) and/or cytotoxic agents. U.S. Pat. No. 6,326,390 B1 (“the '390 patent”) discloses treatment options including use of adenosine A₃ receptor antagonists in combination with anti-angiogenic and/or cytotoxic agents to inhibit the growth of tumors. The '390 patent, which is hereby incorporated by reference in its entirety, discloses A₃ receptor antagonists, anti-angiogenic agents, and cytotoxic agents useful for practicing this embodiment.

In specific embodiments of this aspect, the treatment options include administration of an adenosine A₃ receptor antagonist in combination with a cytotoxic agent to treat patients suffering from multi-drug resistant cancers. For instance, United States Patent Application Publication No. 2004/0067932 A1, hereby incorporated by reference in its entirety, discloses methods of synergistically enhancing the chemotherapeutic treatment of cancer (including human colon carcinomas) by administering an effective amount of a high affinity adenosine A₃ receptor antagonists either prior to or during administration of a chemotherapeutic cancer agent.

In another aspect, the methods comprise determining that a patient has a cancer that is likely to respond to treatment with an A₃ receptor antagonist, then advising a medical professional to treat the patient with an effective amount of an A₃ receptor antagonist. In certain embodiments, the cancer can be of the solid tumor type or melanoma type. In specific embodiments of this aspect, the treatment may include administering to the patient the A₃ receptor antagonist with other anti-tumor agents, such as anti-angiogenic agents (including adenosine A₂ₐ antagonists) and/or cytotoxic agents as described above. In a specific embodiments of this aspect, the treatment may include administering to the patient the A₃ receptor antagonist in combination with a cytotoxic agent to treat patients suffering from multi-drug resistant cancers as described above.

In another aspect, the methods comprise determining that a patient has a pre-cancerous condition for cancer that is likely to respond to treatment with an A₃ receptor antagonist, then advising a medical professional of the treatment option of administering to the patient an effective amount of an A₃ receptor antagonist. In certain embodiments, the cancer can be of the solid tumor type or melanoma type. In specific embodiments of this aspect, the treatment option may include administering to the patient the A₃ receptor antagonist with other anti-tumor agents, such as anti-angiogenic agents (including adenosine A₂ₐ antagonists) and/or cytotoxic agents as described above. In a specific embodiments of this aspect, the treatment options include administration of an adenosine A₃ receptor antagonist in combination with a cytotoxic agent to treat patients suffering from multi-drug resistant cancers as described above.

The following examples are given for the purpose of illustrating various embodiments of the invention and are not meant to limit the present invention in any fashion.

### EXAMPLES

Role of Adenosine A₃ Colorectal Cancer

The possibility that adenosine plays a role in the progression of cancer has aroused considerable interest in recent years (29-31). Since the observation that adenosine could be detected in the interstitial fluid surrounding a carcinoma (4), several reports have shown the effects and the possible mechanism of action of this nucleoside on tumor cell growth (11, 12, 32). However, despite the fact that several studies seem to indicate an emerging role for the A₃ receptor as a good candidate for the identification of tumor cells (16-19), comparative studies performed on normal and tumor tissues are not currently available. The studies leading to the present invention provide the first extensive analysis of the presence of A₃ adenosine receptors in carcinomatous tissue and paired adjacent/remote normal mucosa taken from 73 donors undergoing surgical treatment for colorectal carcinoma. By means of receptor binding studies, A₃ protein was identified in all normal and malignant tissue samples. However, the amount of A₃ receptors was elevated by 2-fold or more in primary colon carcinomas as compared with adjacent and remote normal mucosa, respectively (P<0.05). To verify that the overexpression of A₃ protein was specifically due to neoplastic cells, considering that colonic tumor tissues include neutrophils and other infiltrating cells, immunocytochemical studies were performed. The tumors analyzed showed a strong immunoreactivity at the level of neoplastic epithelial cells and a variable degree of positive antibody staining in smooth muscle and inflammatory cells. In contrast the epithelial cells of corresponding normal colonic mucosa showed generally weak immunoreactivity in agreement with previous observations obtained in human colon mucosa (33). The overexpression of A₃ receptors in colorectal tumors raises the question as to the underlying mechanism, in particular whether this phenomenon is due to an increase in A₃ receptor gene expression. In most cases the drastic differences in A₃ protein levels between malignant and non-malignant colon tissue are not due to changes in the corresponding mRNA. Although we can not completely rule out the possibility that A₃ mRNA is rapidly degraded under in vivo conditions in tumors but not in normal colon tissue, it appears more likely that post-transcriptional control mechanisms are involved in the upregulation of the amount of A₃ receptor protein in tumors.

Protein expression data revealed broad inter-individual variations both in normal and neoplastic tissues. By correlating tumor-to-mucosa A₃ receptor ratio with various clinical parameters, we found a tendency for a lower protein expression in less advanced (stages I-II) compared to more advanced tumor stages (III-IV), which was statistically significant using the T/M ratio of 2 to define low from high expression (P<0.001).

Furthermore, there was a tendency toward a more pronounced expression of A₃ receptors in larger adenomas than small size adenomas suggesting that the expression level of this adenosine receptor subtype may reflect the adenoma-carcinoma sequence. In light of these results, it is possible that A₃ protein could be required during all stages of cancer development with a major role in cancer aggressiveness. The possibility of identifying a diagnostic and prognostic cancer index is suggested by our observations in peripheral circulating blood cells. Indeed, several studies have compared receptor expression profiles in tissues and peripheral blood cells from normal and pathological conditions and found a positive association or trend (22-24). In the present study we found that both peripheral lymphocytes and neutrophils obtained from 30 colorectal cancer patients showed a greater than 3-fold overexpression of A₃ receptors compared with blood cells from healthy donors, in line with the data found in tissues (P<0.01). We did not find any association with stage, tumor site, patient age or gender. The mechanisms of this upregulation are not known. However it is interesting that, binding parameters in tissues as in circulating blood cells discriminate between small size adenomas and cancer suggesting this protein may be a requirement of colorectal tumor progression. Consistent with these data we found also that A₃ receptors of circulating blood cells, in a small cohort of subjects, normalizes after surgical treatment, and in accord with the negative results of follow-up exams including CEA antigen, CT Scan and colonoscopy. Hence, the good-health of patients after surgical re-section seems to be associated with a restoration of a normal adenosinergic system, at least in terms of the A₃ receptor expression. These findings will be used for clinical applications. In particular, a potential role in the screening of high risk individuals or in the follow-up of patients after surgical resection could be evaluated by examination of neutrophil A₃ expression for example, in addition to CEA determination.

These studies leading to the present invention provide the first evidence, that the A₃ receptor plays a role in human colon tumor development, and more importantly, it can potentially be used as a diagnostic marker or as a therapeutic target of Adenosine A₃ receptor ligands for colon cancer treatment.

The following materials and methods were used in the Examples:

Materials. [³H]MRE 3008F20 (specific activity 67 Ci/mmol) was synthesized at Amersham International (Buckinghamshire, UK).

[³H]DPCPX (specific activity 120 Ci/mmol) was purchased from NEN Research Products (Boston, Mass; USA). [³H]ZM 241385 (specific activity 17 Ci/mmol) was obtained from Tocris Cookson Ltd (Bristol, UK). The A₃ antibody was purchased from Alpha Diagnostic (S. Antonio, Tex., USA). TaqMan MGB probe and A₃ primers were obtained from Applied Biosystems (UK). All other reagents were of analytical grade and obtained from commercial sources.

Patients and Tissues. All patients included in the study had procedures performed at the St. Anna Ferrara University Hospital, between September 2001 and 2002. The protocol was approved by the local Ethics Committee and informed consent was obtained from each patient. 77 subjects who underwent surgical resection for colorectal adenocarcinomas (N=73, primary tumors, with a median age of 70±10 years) or large adenomas with severe dysplasia (>2 cm in diameter, N=4 with a median age of 67±13 years) were included in the study. A further 4 small adenomas (<1 cm in diameter, with a median age of 65±11 years) which were endoscopically removed from 4 patients have also been examined. When the surgical specimens were obtained, paired samples of tumor and surrounding peritumoral normal mucosa at a distance of 2 cm (adjacent normal mucosa) and 10 cm (remote normal mucosa) from the tumor were prepared. The specimens were examined by means of receptor binding, immunocytochemistry and real-time RT-PCR studies. All tumors were histologically typed and graded. The characteristics of the tumors, including sex and age, location, histologic type, degree of differentiation and tumor stage, of each patient are reported in Table 1.

PREPARATION OF COLONIC MEMBRANE SUSPENSIONS. Colon cancer tissues and related mucosa were homogenized in PBS buffer with a Polytron (Kinematica) and centrifuged for 15 min at 48,000 g. The membrane pellet was resuspended in 50 mM Tris/HCl buffer pH 7.4 (50 mM Tris/HCl, 10 mM MgCl₂, 1 mM EDTA) and incubated with 3 IU/ml of adenosine deaminase for 30 min at 37° C. This suspension was used for binding experiments.

Preparation of Blood Peripheral Membrane Suspensions. Neutrophils and lymphocytes, isolated from peripheral venous blood samples (40 ml), were obtained from patients with colorectal cancer undergoing surgery and from healthy volunteers. Membranes from neutrophils and lymphocytes were prepared as previously described (19, 24) and were used for binding experiments.

[³H]MRE 3008F20 Binding Assays. [³H]MRE 3008F20, 5-N-(4-methoxyphenyl-carbamoyl)amino-8-propyl-2(2furyl)-pyrazolo-[4,3 e]-1,2,4-triazolo [1,5-c]pyrimidine ([³H]MRE 3008F20) is a potent and selective A₃ receptor ligand (25). In saturation experiments, membrane homogenates (80-100 μg of protein/assay) were incubated in duplicate with 10-12 different concentrations of [³H]MRE 3008F20. Non-specific binding was determined in the presence of 1 μM MRE 3008F20. Bound and free radioactivity were separated, after an incubation time of 120 min at 4° C., by filtering the assay mixture using a Brandel cell harvester. The protein concentration was determined according to a Bio Rad method (26). Binding to A₁ and A_{2A} adenosine receptors was performed using [³H]1,3-dipropyl-8-cyclopentyl-xanthine ([³H]DPCPX) and [³H]4-(2-[7-amino-2-(2-furyl)-[1,2,4]triazolo-[2,32][1,3,6]triazinyl-amino]ethyl)-phenol ([³H]ZM 241385), respectively, as previously described (16, 24).

Immunocytochemistry of Colonic Tissues. Sections (5 μm), obtained from formalin-fixed paraffin-embedded tissue blocks of 5 colorectal carcinomas and corresponding adjacent and remote normal mucosa, were routinely deparaffinized, rehydrated, and rinsed in PBS. Sections were then incubated overnight at 4° C. with polyclonal rabbit antibody against human A₃ receptor (1:100 in PBS). An UltraVision streptavidin-biotin peroxidase detection kit (TP-060-HL; Lab Vision Corporation, Fremont, Calif.) was employed as the secondary detection system, and the peroxidase reaction was developed using diaminobenzidine tetrachloride as chromogen. Finally, slides were lightly counterstained with Mayer hematoxylin.

Real-Time RT-PCR experiments. Tissue samples, obtained from specimens of colorectal cancer and remote normal tissues, were removed immediately and put into TRIzol reagent for RNA extraction. Quantitative real-time RT-PCR assay (27) of A₃ mRNA transcript was carried out using a gene-specific double fluorescently labelled TaqMan MGB probe (minor groove binder) and ABI Prism 7700 Sequence Detection System (Applied Biosystems, Warrington Cheshire, UK). The following primer and probe sequences were used for real-time RT-PCR: A₃ forward primer, 5′-ATGCCTTTGGCCATTGTTG-3′; A₃ reverse primer 5′-ACAATCCACTTCTACAGCTGCCT-3′; A₃ MGB probe 5′-FAM-TCAGCCTGGGCATC-TAMRA-3′ in which the fluorescent reporter FAM and the quencher TAMRA are 6-carboxy fluorescein and 6-carboxy-N,N,N′,N′-tetramethylrhodamine, respectively. For the real-time RT-PCR of the reference control gene, human β-actin kits were used, and the probe was fluorescent-labeled with VIC™ (Applied Biosystems, Monza, Italy).

Data and statistical analysis. A weighted nonlinear least squares curve fitting program, LIGAND was used for computer analysis of the data from saturation experiments (28). Significant differences between control and patients with colorectal cancer were assessed using Student's t-test or analysis of variance (ANOVA) and the Dunnett's test when required. P<0.05 was considered significant. All data are reported as means±SEM.

### Example 1

### Overexpression of A₃ Adenosine Receptor Subtype in Patients with Colorectal Cancer

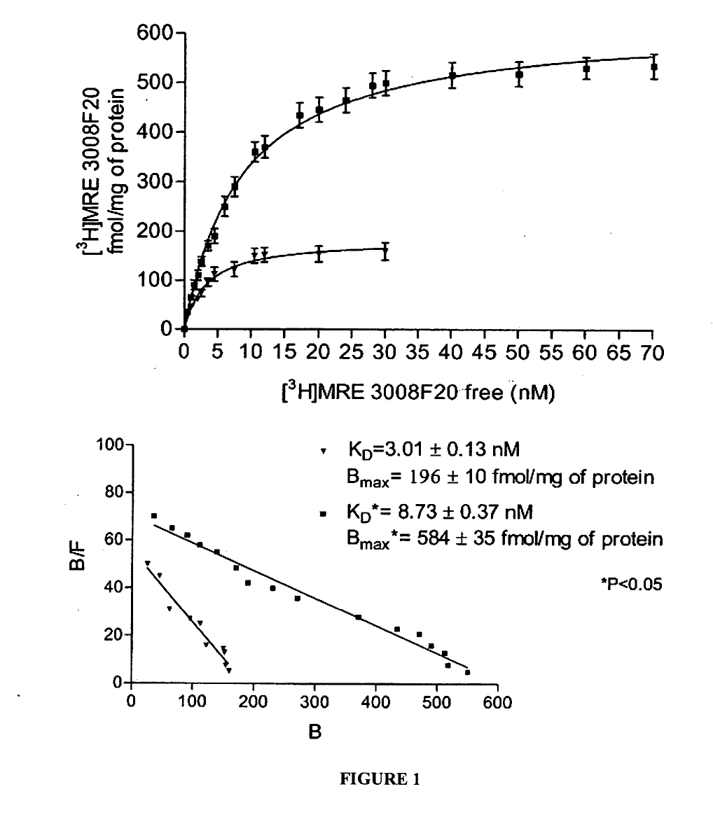

To address the question as to the putative relevance of A₃ receptor protein in human carcinogenesis and tumor progression, we analysed the presence of A₃ receptors in 73 primary colorectal carcinomas compared to adjacent normal (AN) and remote normal (RN) mucosa from the same individual. As shown in table 2, receptor binding parameters of the A₃ ligand [³H]MRE 3008F20, were significantly (P<0.05) higher in tumor tissues (K_{D}=8.73±0.37 nM; Bₘₐₓ=584±35 fmol/mg of protein) when compared with the corresponding values in AN mucosa (K_{D}=4.56±0.21 nM; Bₘₐₓ=295±20 fmol/mg of protein) and with those found in RN mucosa (K_{D}=3.01±0.13 nM; Bₘₐₓ=196±10 fmol/mg of protein). To establish the basis for comparison of tumors with RN mucosa, we first examined A₃ receptors in colonic membranes derived from 14 patients operated for non-neoplastic large bowel disease; affinity and density values were found to be statistically not different from those found in RN mucosa and different from the tumor (K_{D}=2.93±0.19 nM; Bₘₐₓ=163±10 fmol/mg of protein) (P<0.05). Therefore, in order to take into account possible inter-individual variations in the expression level of A₃ receptors, tumor tissue and the corresponding normal tissue obtained from the same patient were compared (FIG. 1). The inter-individual variability of A₃ receptor density in the tumor samples was about 2-4 fold. Moreover, the mean ratio of tumor/remote mucosa A₃ receptor concentration (T/M ratio) was 3 fold and 20 of 73 human colon carcinomas showed a more than 4-fold overexpression of A₃ protein compared with the RN mucosa of the same donor. The T/M ratio of A₃ receptor expression was examined in relation to TNM stage, which is used as the most relevant prognostic clinical parameter. Although the T/M ratio of A₃ receptors did not significantly correlate to TNM stage (I-IV), we observed that early stage tumors (I and II) ( ) had a greater frequency of T/M ratio <2 than advanced stage (III, IV) tumors (stage I, 4/4<2; stage II, 13/33<2; stage III, 3/28<2; stage IV, 1/8<2) (χ² test, P<0.001). To further investigate if the A₃ receptor status might reflect a progression in the malignancy, binding parameters were evaluated in 4 small adenomas with low-grade dysplasia and 4 large adenomas with high-grade dysplasia. The small adenomas had affinity and density values very similar to the mucosa of healthy subjects (K_{D}=2.99±0.50 nM; Bₘₐₓ=155±24 fmol/mg of protein, N=4) whereas the large adenomas showed increased binding parameters (K_{D}=6.3±0.8 nM; Bₘₐₓ=302±15 fmol/mg of protein, N=4, P<0.05), suggesting a tendency toward a greater A₃ receptor expression during colorectal tumor progression. Finally, no statistically significant correlation (χ² test) was observed by comparing T/M A₃ receptor ratio with the degree of tumor differentiation or tumor location (P>0.3).

### Example II

### Overexpression of A₃ Adenosine Receptor Subtype in Peripheral Blood Cells of Patients with Colorectal Cancer and Normalization after Surical Resection for Colorectal Cancer

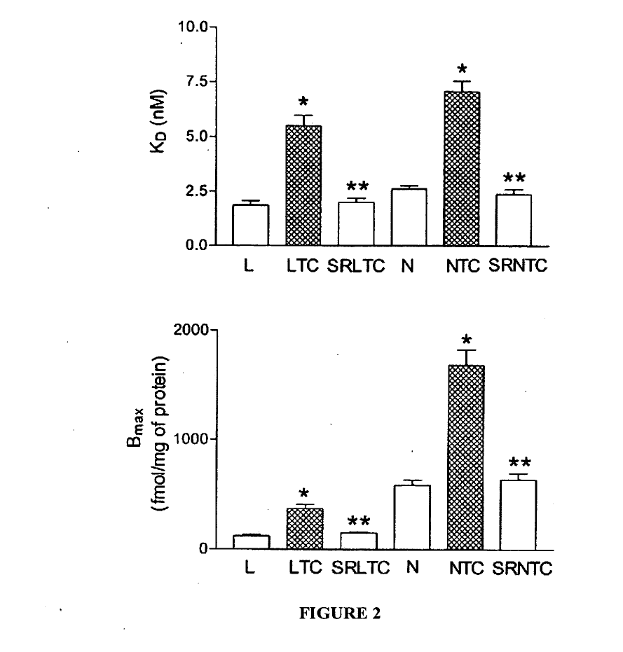

In order to evaluate the possibility of A₃ receptor alterations in peripheral blood cells of patients with colorectal cancer we examined the K_{D} and Bₘₐₓ values of A₃ binding sites in neutrophils and lymphocytes of patients affected by colorectal cancer. Binding parameters reported in table 3 were approximately 3-fold higher as compared with those found in healthy subjects (P<0.01) indicating an increase in receptor number and an affinity decrease that was in agreement with the variations found in the cancer tissues (FIG. 2). Moreover, binding data obtained from 3 patients with small adenomas were similar to those found in healthy subjects (K_{D}=2.46±0.52, 2.67±0.29 nM and Bmax=106±15, 547±77 fmol/mg of protein, N=3 in lymphocytes and neutrophils, respectively) suggesting that a progression in the malignancy may be reflected by binding data obtained in blood cells. In contrast, statistical analysis of A₃ receptor binding parameters and TNM stage did not reveal a significant correlation.

In a cohort of 10 patients, the peripheral A₃ receptor expression was studied 12 months from colorectal surgical resection. K_{D} and Bmax of A₃ receptors on neutrophils and lymphocytes returned to normal values (FIG. 2 and Table 3), and were in accord with the negative results of follow-up examinations, including human carcinoembryonic antigen (CEA), computed tomography imaging (CT Scan) and colonoscopy, indicating the absence of tumor recurrence.

### Example III

### Immunocytochemistry of Colonic Tissues

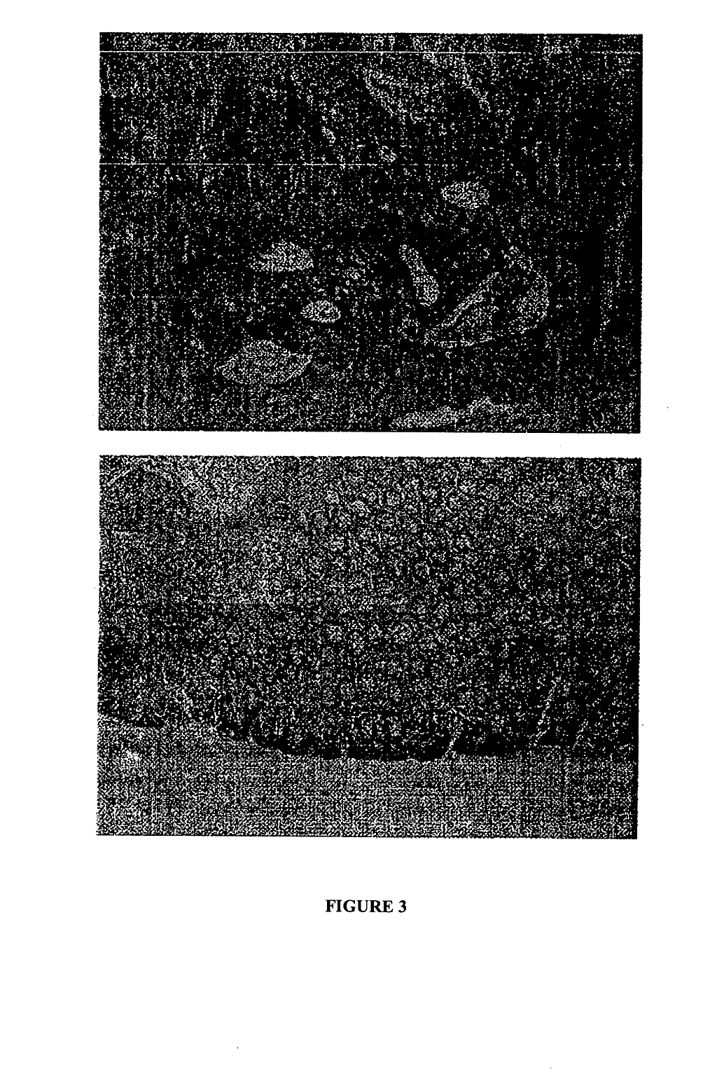

Colonic tissues consist of several types of cells including epithelial and fibroblast cells, nerve ganglia, as well as endothelial and smooth muscle cells of blood vessels. In order to assess the specificity of A₃ overexpression in tumor cells, the distribution of these receptors was evaluated in paired tumor and RN mucosa sections. Immunohistochemical analysis with anti-A₃ adenosine receptor antibody demonstrated strong cytoplasmatic and cell membrane staining of the majority of neoplastic cells in the 5 colorectal carcinomas examined. Corresponding normal colonic mucosa, taken at a distance from the tumor, showed generally weak immunoreactivity in the cytoplasm and cytoplasmatic membrane of epithelial cells. FIG. 3 shows the results obtained in a moderately differentiated adenocarcinoma (upper panel) and in the related RN mucosa (Lower panel). Finally, positive immunostaining was observed in the nervous structures of the bowel wall, whereas a variable degree of immunoreactivity was detected in smooth muscle cells, inflammatory and stromal cells.

### Example IV

### Expression of A₃ Receptor Gene

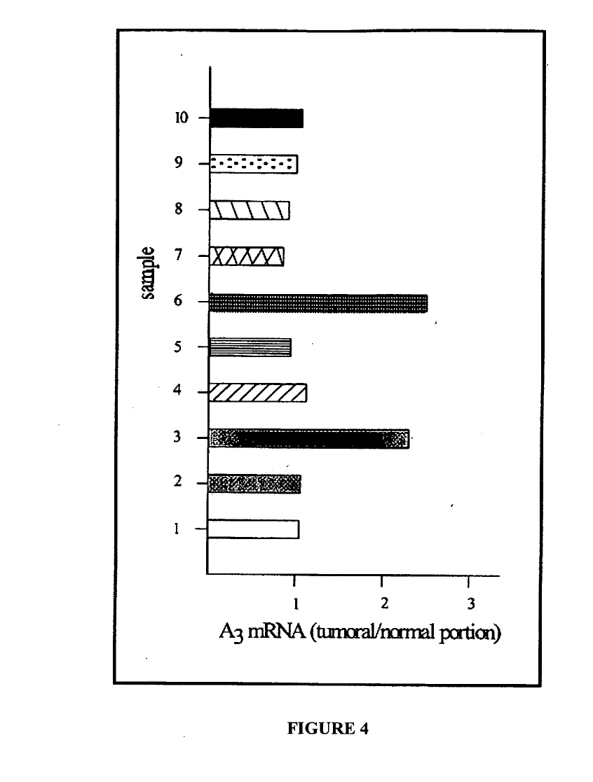

The level of expression of A₃ receptor protein in tumors raises the question as to the underlying mechanism. One possibility is that A₃ receptor mRNA content is elevated in tumors. Therefore, A₃ receptor mRNA content was investigated on RNA from 10 paired tumor and normal tissue samples obtained from colon cancer patients by use of real-time quantitative RT-PCR. The normalized concentrations of A₃ receptors were determined using β-actin mRNA as endogenous internal control; the mRNA content was expressed as A₃ receptor mRNA/β-actin mRNA. The overexpression of A₃ receptor protein in tumors was not reflected by the specific A₃ mRNA transcription increase. As shown in FIG. 4, we found an increased content of A₃ receptor mRNA in two samples, in which a 2-fold increase was found with respect to the corresponding normal portion. In the majority of cases the content of A₃ receptor mRNA was not different from that of the corresponding non-malignant tissue. These data suggest that the elevated expression of A₃ receptor protein in colon cancer was not essentially associated to an increase in A₃ receptors mRNA.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

It is understood that this invention is not limited to the particular materials and methods described herein. It is also to be understood that the terminology used herein is for the purpose of describing particular embodiments and is not intended to limit the scope of the present invention. As used herein, the singular forms “a”, “an”, and “the” include plural reference unless the context clearly dictates otherwise. For example, a reference to “a leukocyte cell” includes a plurality of such leukocyte cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

### REFERENCES

1. Fredholm, B. B., Ijzerman, A. P., Jacobson, K. A., Klotz, K. N., and Linden, J. International union of pharmacology. XXV. Nomenclature and classification of adenosine receptors. Pharmacol. Rev., 53:527-552, 2001.

2. Linden, J. Molecular approach to adenosine receptors: receptor-mediated mechanisms of tissue protection. Annu. Rev. Pharm. Toxicol. 41: 775-787, 2001.

3. Poulsen, S. A., and Quinn, R. J. Adenosine receptors: new opportunities for future drugs. Bioorg. Med. Chem., 6:619-641, 1998.

4. Blay, J., White, T. D., and Hoskin, D. W. The extracellular fluid of solid carcinoma contains immunosuppressive concentrations of adenosine. Cancer Res., 57:2602-2605, 1997.

5. MacKenzie, W., Hoskin, D. W., and Blay, J. Adenosine inhibits the adhesion of anti-CD3-activated killer lymphocytes to adenocarcinoma cells through an A₃ receptor. Cancer Res., 54:3521-3526, 1994.

6. Ohta, A., and Sitkovsky, M. Role of G-protein-coupled receptors in downregulation of inflammation and protection from tissue damage. Nature, 414:916-920, 2001.

7. Feoktistov, I., Goldstein, A. E., Ryzhov, S., Zeng, D., Belardinelli, L., Voyno-Yasenetskaya, T., and Biaggioni, I. Differential expression of adenosine receptors in human endothelial cells: role of A_{2B} receptors in angiogenic factor regulation. Circ. Res., 90:531-538, 2002.

8. Abbracchio, M. P., Rainaldi G., Giammarioli, A. M., Ceruti, S., Brambilla, R., Cattabeni, F., Barbieri, D., Franceschi, C., Jacobson, K. A., Malorni, W. The A₃ adenosine receptor mediates cell spreading, reorganization of actin cytoskeleton, and distribution of BcI-XL: studies in human astroglioma cells. Biochem. Byophys. Res. Comm., 241:297-304, 1997.

9. Brambilla, R., Cattabeni, F., Ceruti, S., Barbieri, D., Franceschi, C., Kim, Y., Jacobson, K. A., Klotz, K. N., Lohse, and Abbracchio, M. P. Activation of the A₃ adenosine receptor affects cell cycle progression and cell growth. Naunyn-Schmiedeberg's Arch. Pharmacol., 361: 225-234, 2000.

10. Fishman, P., Bar-Yehuda, S., and Vagman, L. Adenosine and other low molecular weight factors released by muscle cells inhibit tumor cell growth. Cancer Res., 58:3181-3187, 1998.

11. Ohana, G., Bar-Yehuda, S., Barer, F., and Fishman, P. Differential effect of adenosine on tumor and normal cell growth: focus on the A₃ adenosine receptor. J. Cell. Physiol., 186:19-23, 2001.

12. Merighi, S., Mirandola, P., Milani, D., Varani, K., Gessi, S., Klotz, K-N., Leung, E., Baraldi, P. G., and Borea, P. A. Adenosine receptors as mediators of both cell proliferation and cell death of cultured human melanoma cells. J. Invest. Dermatol., 119:923-933, 2002.

13. Peyot, M. L., Gadeau, A. P., Dandre, F., Belloc, I., Dupuch, F., and Desgranges, C. Extracellular adenosine induces apoptosis of human arterial smooth muscle cells via A_{2B} purinoceptor. Circ. Res., 86:76-85, 2000.

14. Fishman, P., Bar-Yehuda, S., Madi, L., and Cohn, I. A₃ adenosine receptor as a target for cancer therapy. Anticancer drugs, 13:1-8, 2002.

15. Ohana, G., Bar-Yehuda, S., Arich, A., Madi, L., Dreznick, Z., Rath-Wolfson, L., Silberman, D., Slosman, G., Fishman, P. Inhibition of primary colon carcinoma growth and liver metastasis by the A₃ adenosine receptor agonist CF101. Br. J. Cancer, 89:1552-1558.

16. Gessi, S., Varani, K., Merighi, S., Morelli, A., Ferrari, D., Leung, E., Baraldi, P. G., Spalluto, G., and Borea, P. A. Pharmacological and biochemical characterization of A₃ adenosine receptors in Jurkat T cells. Br. J. Pharmacol., 134:116-126, 2001.

17. Merighi, S., Varani, K., Gessi, S., Cattabriga, E., lannotta, V., Ulouglu, C., Leung, E., and Borea, P. A. Pharmacological and biochemical characterization of adenosine receptors in the human malignant melanoma A375. Br. J. Pharmacol., 134:1215-1226, 2001.

18. Suh, B. C., Kim, T. D., Lee, J. U., Seong, J. K., and Kim, K. T. Pharmacological characterization of adenosine receptors in PGT-beta mouse pineal gland tumour cells. Br. J. Pharmacol., 134:132-142, 2001.

19. Gessi, S., Varani, K., Merighi, S., Cattabriga, E., Iannotta, V., Leung, E., Baraldi, P. G., and Borea, P A. A₃ adenosine receptors in human neutrophils and promyelocytic HL60 cells: a pharmacological and biochemical study. Mol. Pharmacol., 61:415-424, 2002.

20. Fishman, P., Bar-Yehuda, S., Barer, F., Madi, L., Multani, A. S., and Pathak, S. The A₃ adenosine receptor as a new target for cancer therapy and chemoprotection. Exp. Cell. Res., 269:230-236, 2002.

21. Hellmich, M. R., Rui, X-L., Hellmich, H. L., Declan Fleming, R. Y., B M Evers, and Townsend, C. M. Jr. Human colorectal cancers express a constitutively active cholecystochinin-B/gastrin receptor that stimulates cell growth. J. Biol. Chem., 275:32122-32128, 2000.

22. Brodde, O. E., Beckering, J. J., and Michel, M. C. Human heart β-adrenoceptors: a fair comparison with lymphocyte β-adrenoceptors? Trends Pharmacol. Sci., 8:403-4071, 1987.

23. Varani, K., Gessi, S., Caiazza, A., Rastelli, G., Portaluppi, F., and Borea, P. A. Platelet α₂-adrenoceptor alterations in patients with essential hypertension. Br. J. Clin. Pharmacol., 47:167-172, 1999.

24. Varani, K., Laghi-Pasini, F., Camurri, A., Capecchi, P. L., Maccherini, M., Diciolla, F., Ceccatelli, L., Lazzerini, P. E., Ulouglu, C., Cattabeni, F., Borea, P. A., and Abbracchio, M. P. Changes of peripheral A_{2A} adenosine receptors in chronic heart failure and cardiac transplantation. Faseb J., 17:280-282, 2002.

25. Varani, K., Merighi, S., Gessi, S., Klotz, K-N., Leung, E., Baraldi, P. G., Cacciari, B., Romagnoli, R., Spalluto, G., and Borea, P. A. [³H]MRE 3008F20: a novel antagonist radioligand for the pharmacological and biochemical characterization of human A₃ adenosine receptors. Mol. Pharmacol., 57:968-975, 2000.

26. Bradford, M. M. A rapid and sensitive method for the quantification of microgram quantities of protein utilizing the principle of protein dye-binding. Anal. Biochem., 72:248-254, 1976.

27. Higuchi, R., Fockler, C., Dollinger, G., and Watson, R. Kinetic PCR analysis: real-time monitoring of DNA amplification reactions. Biotechnology, 11:1026-1030, 1993.

28. Munson, P. J., and Rodbard, D. Ligand: a versatile computerized approach for the characterization of ligand binding systems. Anal. Biochem., 107:220-239, 1980.

29. Merighi, S., Mirandola, P., Varani, K., Gessi, S., Leung, E., Baraldi, P. G., Tabrizi, M. A., and Borea, P. A. A glance at adenosine receptors: novel target for antitumor therapy. Pharmacol & Ther in press.

30. Fishman, P., Madi, L., Bar-Yehuda, S., Barer, F., Del Valle, L., and Khalili, K. Evidence for involvement of Wnt signaling pathway in IB-MECA mediated suppression of melanoma cells. Oncogene, 21:4060-4064, 2002.

31. Bar-Yehuda, S., Barer, F., Volfsson, L., and Fishman, P. Resistance of muscle to tumor metastases: a role for A₃ adenosine receptor agonists. Neoplasia, 3:125-131, 2001.

32. Barry, C. P., and Lind, S. E. Adenosine-mediated killing of cultured epithelial cancer cells. Cancer Res., 60:1887-1894, 2000.

33. Christofi, F. L., Zhang, H., Yu, J., Guzman, J., Xue, J., Kim, M., Wang, Y-Z., Cooke, H. J. Differential gene expression of adenosine A₁, A_{2A}, A_{2B} and A₃ receptors in the human enteric nervous system. J. of Comparative Neurology, 439:46-64, 2001.

We claim:

1. A method for the diagnosis of cancer in a mammal, comprising
(a) obtaining a biological sample from the mammal; and
(b) detecting overexpression of A₃ receptor comprising the polypeptide of SEQ ID NO: 1 in the sample compared to the level in a healthy mammal, wherein said increased level is diagnostic of having cancer in the mammal.

2. The method of claim 1, wherein the biological sample is leukocytes from blood.

3. The method of claim 1, wherein the cancer is a solid tumor.

4. The method of claim 3, wherein the solid tumor is pancreatic carcinoma, breast carcinoma, prostate carcinoma, colorectal carcinoma, lung carcinoma or ovarian carcinoma.

5. The method of claim 3, wherein the biological sample is tissue biopsied from a body site suspected of containing the tumor.

6. The method of claim 1, wherein the cancer is melanoma.

7. The method of claim 6, wherein the biological sample is skin tissue.

8. The method of claim 1, wherein over expression A₃ receptor is detected using a receptor binding assay.

9. The method of claim 1, wherein over expression A₃ receptor is detected using an immunocytochemical assay.

10. The method of claim 1, wherein over expression A₃ receptor is detected using flow cytometric techniques.

11. The method of claim 1, wherein over expression A₃ receptor is detected using RT-PCR.

12. A method for the diagnosis of cancer in a mammal, comprising
(a) obtaining a biological sample from the mammal; and
(b) detecting an overexpression of A₃ receptor protein in the sample compared to the level in a healthy animal by using a detectably labeled antibody that specifically binds the polypeptide of SEQ ID NO: 1 wherein said increased level is diagnostic of cancer in the mammal.

13. The method of claim 12, wherein the biological sample is leukocytes from blood.

14. The method of claim 12, wherein the cancer is a solid tumor.

15. The method of claim 14, wherein the solid tumor is pancreatic carcinoma, breast carcinoma, prostate carcinoma, colorectal carcinoma, lung carcinoma or ovarian carcinoma.

16. The method of claim 14, wherein the biological sample is tissue biopsied from a body site suspected of containing the tumor.

17. The method of claim 12, wherein the cancer is melanoma.

18. The method of claim 17, wherein the biological sample is skin tissue.

19. A method for the diagnosis of cancer in a mammal, comprising
(a) obtaining a biological sample from the mammal; and
(b) detecting an overexpression of A₃ receptor protein in the sample compared to the level in a healthy mammal by using a detectably labeled ligand that specifically binds the polypeptide of SEQ ID NO: 1 wherein said increased level is diagnostic of cancer in the mammal.

20. The method of claim 19, wherein the biological sample is leukocytes from blood.

21. The method of claim 19, wherein the cancer is a solid tumor.

22. The method of claim 21, wherein the solid tumor is pancreatic carcinoma, breast carcinoma, prostate carcinoma, colorectal carcinoma, lung carcinoma or ovarian carcinoma.

23. The method of claim 21, wherein the biological sample is tissue biopsied from a body site suspected of containing the tumor.

24. The method of claim 19, wherein the cancer is melanoma.

25. The method of claim 24, wherein the biological sample is skin tissue.

26. A method for diagnosing and treating a patient for cancer, the method comprising determining that the patient has a cancer that is likely to respond to treatment with an A₃ receptor antagonist, then advising a medical professional of the treatment option comprising administering to the patient an effective amount of an A₃ receptor antagonist.

27. The method of claim 26, wherein the treatment option further comprises administering to the patient an effective amount of an agent selected from an anti-angiogenic agent, a cytotoxic agent or combination thereof.

28. A method for diagnosing and treating a patient for cancer, the method comprising determining that the patient has a cancer that is likely to respond to treatment with an A₃ receptor antagonist, then advising a medical professional to treat the patient with an effective amount of an A₃ receptor antagonist.

29. The method of claim 28, wherein the treating further comprises administering to the patient an effective amount of an agent selected from an anti-angiogenic agent, a cytotoxic agent or combination thereof.

30. A kit for the diagnosis of cancer in a patient, wherein the kit comprises an agent that can be used to specifically detect overexpression of A₃ receptor protein in a sample.


SEQUENCE LISTING (5 sequences)

SEQ ID NO: 1 - protein, 318 aa, Homo sapiens
  human adenosine A3 receptor (accession No. P33765)
MPNNSTALSLANVTYITMEIFIGLCAIVGNVLVICVVKLNPSLQTTTFYFIVSLALADIAVGVLVMPLAIVVSLGITIHFYSCLFMTCLLLIFTHASIMSLLAIAVDRYLRVKLTVRYKRVTTHRRIWLALGLCWLVSFLVGLTPMFGWNMKLTSEYHRNVTFLSCQFVSVMRMDYMVYFSFLTWIFIPLVVMCAIYLDIFYIIRNKLSLNLSNSKETGAFYGREFKTAKSLFLVLFLFALSWLPLSIINCIIYFNGEVPQLVLYMGILLSHANSMMNPIVYAYKIKKFKETYLLILKACVVCHPSDSLDTSIEKNSE

SEQ ID NO: 2 - DNA, 19 bp, Artificial Sequence
  A3 forward primer
atgcctttggccattgttg

SEQ ID NO: 3 - DNA, 23 bp, Artificial Sequence
  A3 reverse primer
acaatccacttctacagctgcct

SEQ ID NO: 4 - DNA, 20 bp, Artificial Sequence
  GPI Exon3R primer
tcggtgtagttgatcttctc

SEQ ID NO: 5 - DNA, 14 bp, Artificial Sequence
  A3 MGB probe
  misc_feature 1, 14: fluorescent reporter FAM added at 5′ end and quencher TAMRA (6-carboxy fluorescein and 6-carboxy-N,N,N′,N′-tetramethylrhodamine) added at 3′ end
tcagcctgggcatc